(19) United States
(12) Patent Application Publication
(10) Pub. No.: US 2016/0147002 A1
(43) Pub. Date: May 26, 2016

(54) Light Field-Modulable Optical Needle Assembly

(71) Applicant: LightMed Dental Technology Corp., Kaohsiung City (TW)

(72) Inventors: Yuan-Peng HUANG, Kaohsiung City (TW); Jyh-Rou SZE, Hsinchu City (TW); Hsien-Nan KUO, Kaohsiung City (TW)

(21) Appl. No.: 14/555,437

(22) Filed: Nov. 26, 2014

(51) Int. Cl. F21V 8/00; A61N 5/06

(52) U.S. Cl. CPC G02B 6/0008; A61N 5/0603; A61N 2005/0606

(57) ABSTRACT

This invention relates to a light field-modulable optical needle assembly including a coherent light source, a light conduction member and a light modulation member. The light conduction member includes a light incident face that is proximate to the coherent light source, a light exiting face that is opposite to the light incident face and distal from the coherent light source, and a surrounding face that peripherally extends from the light incident face to the light exiting face to be connected therebetween. The light modulation member is disposed proximate to one of the light incident face, the light exiting face and the surrounding face of the light conduction member and is formed with a microstructure. The light emitted from the light field-modulable optical needle assembly has a light output power distribution adjustable by the microstructure of the light modulation member.

### FIELD OF THE INVENTION

This disclosure relates to an optical needle assembly, more particularly to a light field-modulable optical needle assembly.

### BACKGROUND OF THE DISCLOSURE

Applications of laser beams have prevailed rapidly in recent years. Conventional laser needle devices generally include laser beam sources in combination with optical lenses such as focusing lens and collimating lens, etc., and laser needle tips that are formed of an optical fiber material and that serve as a light conduction member. The conventional laser needle devices are widely applied to laser cosmetic surgeries, laser cutting, laser drilling, laser heat treatments, etc. In particular, the species of the laser needle devices for dental treatments are numerous so as to cope with various conditions. The characteristic differences among the conventional laser needle devices are mainly reflected on diameters, lengths, contours and acting edges of the laser needles, which result in differences in the properties such as sizes, shapes and deflection angles of light spots thus formed by the laser beams and reachable depths in tissues to be treated (e.g., oral tissues in the oral cavity) and so on. The conventional laser needle devices generally have a light output power distribution that conforms to the Gaussian distribution exhibiting a bell-shaped curve and an inhomogeneous intensity distribution. For the light spots generated by the conventional laser needle devices, light output power at the central region of each light spot is higher than that at the peripheral region. Hence, when a conventional laser needle device is applied to perform treatment in the oral cavity, the portion of the oral tissue subjected to the higher light energy output at the central region of the light spot tends to be damaged while the portion of the oral tissue subjected to the lower light energy output at the peripheral region of the light spot tends to have insufficient cutting and sterilization effects.

### SUMMARY OF THE DISCLOSURE

Therefore, the object of the present invention is to provide a light field-modulable optical needle assembly that can alleviate at least one of the aforesaid drawbacks of the prior art.

According to this invention, a light field-modulable optical needle assembly includes: a coherent light source; a light conduction member that has a light incident face which is proximate to the coherent light source, a light exiting face which is opposite to the light incident face and distal from the coherent light source, and a surrounding face which peripherally extends from the light incident face to the light exiting face to be connected therebetween; and a light modulation member that is disposed proximate to one of the light incident face, the light exiting face and the surrounding face of the light conduction member. The light modulation member is formed with a microstructure.

When light is emitted from the coherent light source, the emitted light enters and exits the light conduction member and the light modulation member so as to have a light output power distribution adjusted by the microstructure of the light modulation member.

### BRIEF DESCRIPTION OF THE DRAWINGS

Other features and advantages of the disclosure will become apparent in the following detailed description of the embodiments with reference to the accompanying drawings, of which:

FIG. 1 is a perspective view for illustration of a light conduction member and a light modulation member of the first embodiment of a light field-modulable optical needle assembly according to the present disclosure;

FIG. 2 is a fragmentary sectional view of the first embodiment;

FIG. 3 is a schematic view for illustration of a microstructure of the light modulation member of the first embodiment;

FIG. 4 is a schematic view for illustration of another configuration of the microstructure of the light modulation member of the first embodiment;

FIG. 5 is a perspective view for illustration of a light conduction member and a light modulation member of the second embodiment of a light field-modulable optical needle assembly according to the present disclosure;

FIG. 6 is an exploded perspective view for illustration of a light conduction member and a light modulation member of the third embodiment of a light field-modulable optical needle assembly according to the present disclosure;

FIG. 7 is a fragmentary sectional view of the third embodiment;

FIG. 8 is a fragmentary sectional view for illustration of the fourth embodiment of a light field-modulable optical needle assembly according to the present disclosure;

FIG. 9 is a fragmentary sectional view for illustration of the fifth embodiment of a light field-modulable optical needle assembly according to the present disclosure;

FIG. 10 is a schematic view for illustration of the sixth embodiment of a light field-modulable optical needle assembly according to the present disclosure;

FIG. 11 is a schematic view for illustration of the seventh embodiment of a light field-modulable optical needle assembly according to the present disclosure;

FIG. 12 is a schematic view for illustration of the eighth embodiment of a light field-modulable optical needle assembly according to the present disclosure; and

FIG. 13 is a schematic view for illustration of the ninth embodiment of a light field-modulable optical needle assembly according to the present disclosure.

### DETAILED DESCRIPTION OF THE EMBODIMENTS

Before the present disclosure is described in greater detail with reference to the accompanying embodiments, it should be noted herein that like elements are denoted by the same reference numerals throughout the disclosure.

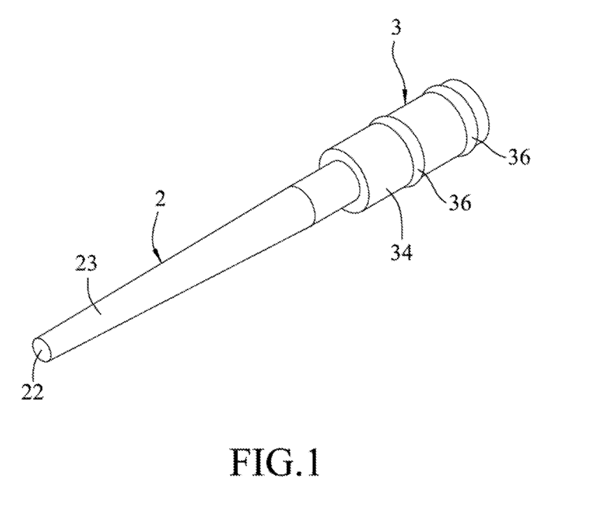

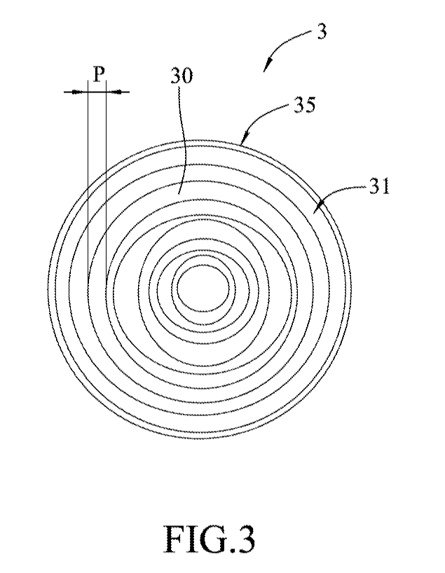

Referring to FIGS. 1 to 3, the first embodiment of a light field-modulable optical needle assembly according to the present disclosure is shown to be adapted for conducting a phototherapy on a site needed to be treated on a human or animal body. The site may be an oral cavity, limbs, a torso, etc. The phototherapy includes but is not limited to debridement of a dental root canal treatment, periodontal disease treatment, laser cutting, skin whitening, debridement of body parts, sterilization, and so on. The light field-modulable optical needle assembly includes a coherent light source 1, a light conduction member 2 and a light modulation member 3.

In this embodiment, laser is taken as an example of the coherent light source 1, such as a laser diode having a central wavelength ranging from about 810 nm to 980 nm, Nd:YAG (neodymium-doped yttrium aluminum garnet) laser having a central wavelength of about 1064 nm, Nd:YAP (neodymium-doped yttrium aluminum perovskite) laser having a central wavelength of about 1340 nm, Er, Cr:YSGG (erbium, chromium:yttrium-scandium-gallium-garnet) laser having a central wavelength of about 2780 nm and Er:YAG (erbium:yttrium-aluminum-garnet) laser having a central wavelength of about 2940 nm. The central wavelength of the coherent light source 1 may be selected based on practical applications and preferably ranges from 800 nm to 3000 nm.

The light conduction member 2 has a central axis (L) and includes a light incident face 21 that is proximate to the coherent light source 1, a light exiting face 22 that is opposite to the light incident face 21 and distal from the coherent light source 1, and a surrounding face 23 that peripherally extends from the light incident face 21 to the light exiting face 22 to be connected therebetween. In this embodiment, both the light incident face 21 and the light exiting face 22 are exemplified but not limited to be perpendicular to the central axis (L). The light conduction member 2 has a pillar-shaped structure, such as a cylindrical or square pillar having a sectional dimension that gradually decreases from the light incident face 21 to the light exiting face 22. The light conduction member 2 may be made from quartz glass or sapphire and may be in the form of optical fibers for transmission of the light emitted from the coherent light source 1.

In this embodiment, the light modulation member 3 is disposed proximate to the light incident face 21 of the light conduction member 2. The light modulation member 3 includes a coupling sleeve 34 tightly coupled one end of the light conduction member 2 that is formed with the light incident face 21, and a light modulation body 35 that is inserted into the coupling sleeve 34 and disposed between the coherent light source 1 and the light incident face 21. The coupling sleeve 34 may be made of a metallic or plastic material and be sleeved by two rubbery O-rings 36 so as to be connectable to a hand-held laser transmission device of a medical laser system. The light modulation body 35 may be made from quartz glass or sapphire. The light modulation body 35 has a first face 31 that faces the coherent light source 1 and is formed with a microstructure 30 having an optical diffraction property. In addition to use of the coupling sleeve 34, the light modulation body 35 may be coupled to the light conduction member 2 with glue, or in any other coupling manners. Hence, the provision of the coupling sleeve 34 is not a requirement for the light field-modulable optical needle assembly of this disclosure. Alternatively, the light modulation member 3 may include only a single component, i.e., the light modulation body 35.

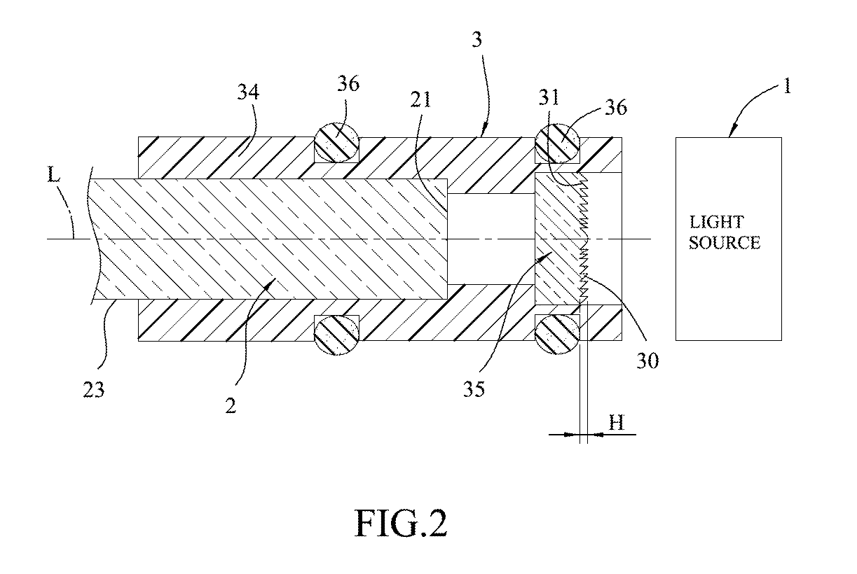

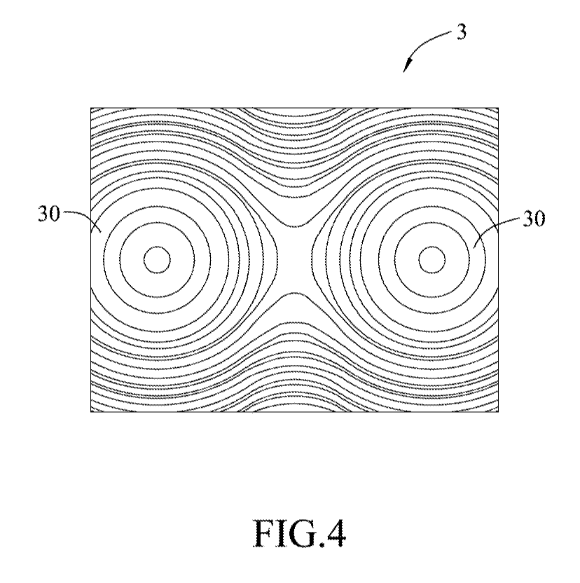

In this embodiment, the microstructure 30 is composed of a plurality of rings that are generally concentric to one another and that have different diameters from each other. As shown in FIG. 2, in the sectional view of the light modulation member 3, the microstructure 30 has a configuration of continuous and alternate crests and troughs. Preferably, the microstructure 30 has structural pitches (P) that range from 0.5 to 200 times of the central wavelength of the coherent light source 1 and structural heights (H) that range from 0.1 to 2000 times of the central wavelength of the coherent light source 1. Each of the structural pitches (P) is defined by a distance between any two adjacent ones of the crests. Each of the structural heights (H) is defined by the distance between one of the troughs to the adjacent one of the crests along the direction of the central axis (L). The ratio between the projected area of the microstructure 30 of the light modulation member 3 on a plane parallel to one of the light incident face 21 and the light exiting face 22 of the light conduction member 2 relative to the area of the one of the light incident face 21 and the light exiting face 22 ranges from 10% to 200%. By means of an arrangement of the structural pitches (P), the structural heights (H) and the surface area ratio of the microstructure 30 in cooperation with the setting of property parameters of the light emitted from the coherent light source 1, intervals, sizes and arrangements of the rings of the microstructure 30 are optimized so that the light emitted from the coherent light source 1 will be diffracted after passing through the microstructure 30. Thereby, the light output power distribution of the light exiting the light field-modulable optical needle assembly is adjusted and the purpose of light modulation is achieved. Moreover, the configuration of the light modulation member 3 is variable, such as having two groups of the concentric rings as shown in FIG. 4, as long as diffraction and modulation of the light emitted from the coherent light source 1 is achievable. For instance, the microstructure 30 may be formed into a pattern composed of a plurality of straight lines or a plurality of dots, or other uneven patterns.

Furthermore, when this embodiment is put into practice, the light emitted from the coherent light source 1 passes through the microstructure 30 of the light modulation member 3 for adjustment of the light output power distribution, and then enters the light conduction member 2 through the light incident face 21 and travels inside the light conduction member 2 before leaving from the light exiting face 22. Specifically, the light emitted from the coherent light source 1 has a light output power distribution that conforms with the Gaussian distribution before passing through the light modulation member 3 and that is adjusted to conform with a flat-top distribution after passing through the light modulation member 3. Light beams having the flat-top distribution may be homogeneous square-shaped light beams, homogeneous circle-shaped light beams, homogeneous line-shaped light beams, etc. The shape of the light beams of the flat-top distribution is determined and adjusted by the configuration of the microstructure 30. After the light passes through the light modulation member 3, the output power and phase of the light are re-distributed to form the required flat-top light beams. The overall output power of the flat-top light beams is homogeneously distributed, and hence the inhomogeneity problem of the conventional optical needle devices, which is caused by the Gaussian distribution with the relatively high output power at the center region and the relatively low output power at the peripheral region, is solved. The light modulation member 3 may serve as a diffraction optical element.

It is noted that in this embodiment, the light emitted from the coherent light source 1 travels inside the light conduction member 2 in the direction of the central axis (L) of the light conduction member 2.

In sum, through the structural design of the light modulation member 3, the light passing through the light modulation member 3 is diffracted so as to achieve the light modulation effect. Hence, when the light field-modulable optical needle assembly is put in practice, the size and shape of the light spot, the light output power distribution and an angle of the light beams are adjustable based on the intended medical treatment. Moreover, the light output has a homogenous distribution so that the subject will be treated with a uniform light power. The problems caused by the phototherapy with the light beams having uneven output power distribution, such as the tissue getting damaged due to reception of the higher light energy output at the central region of the light spot or the tissue having insufficient cutting and sterilization due to reception of the lower light energy output at the peripheral region of the light spot, are solved. As such, the optical needle assembly of this invention can be advantageously applied to the dental laser industry and other phototherapy-related industries involving laser treatments for enhancing convenience and effectiveness of laser treatments.

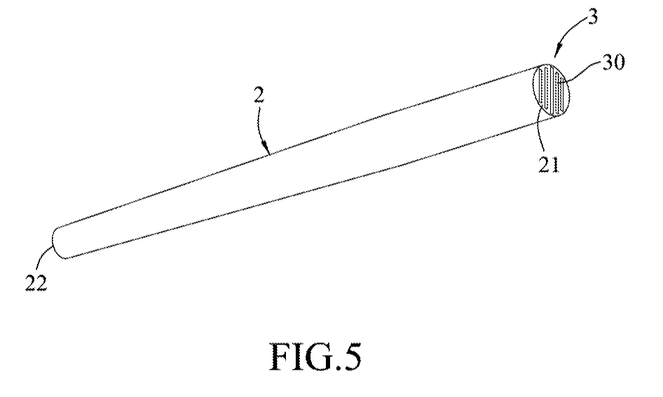

Referring to FIG. 5, the second embodiment of the present disclosure has a structure substantially the same as that of the first embodiment except that the light conduction member 2 and the light modulation member 3 at the second embodiment are integrally formed, and the microstructure 30 of the light modulation member 3 is formed on the light incident face 21 of the light conduction member 2. In this embodiment, the microstructure 30 may be formed by etching the light incident face 21 of the light conduction member 2. The crests and troughs may linearly extend. The troughs may be indentions formed on the light incident face 21 so as to make the light incident face 21 uneven. Alternatively, the microstructure 30 of the light modulation member 3 may be formed on the light exiting face 22 of the light conduction member 2.

It is noted that the light modulation member 3 is adapted to be fabricated through mass production. For instance, a plurality of the light modulation members 3 may be formed at a time by etching a substrate to form a plurality of the microstructures 30 and then cutting the substrate to separate the microstructures 30 from one another. The light modulation members 3 thus formed are respectively bonded to a plurality of the light conduction members 2 so as to form a plurality of the optical needles.

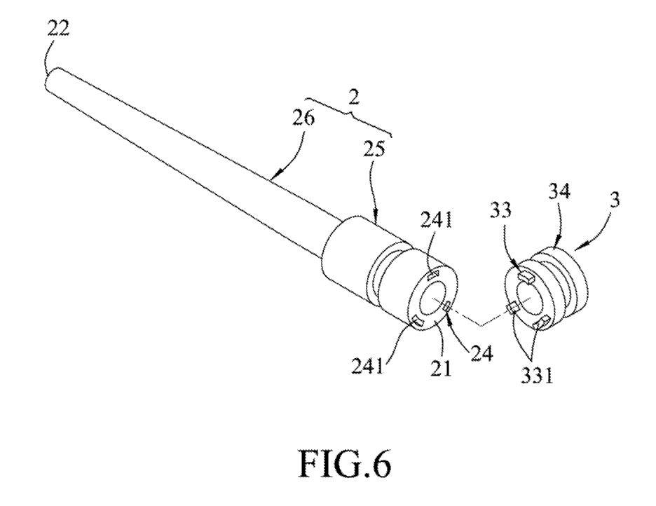

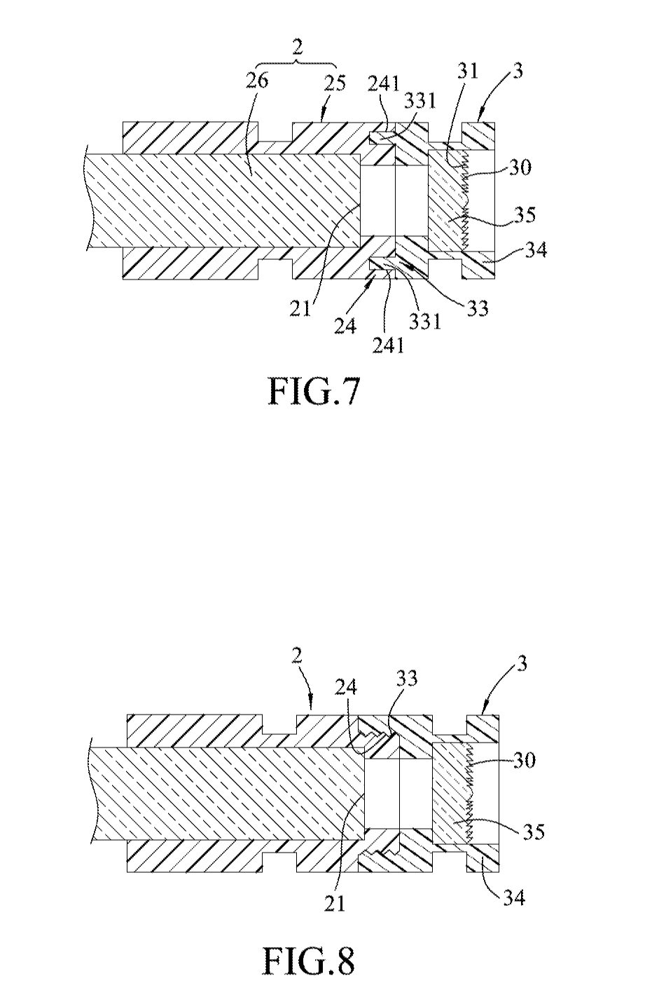

Referring to FIGS. 6 and 7, the third embodiment of the present disclosure has a structure substantially the same as that of the first embodiment. However, the light conduction member 2 further has a first engaging portion 24 that is disposed proximate to the light incident face 21. The light modulation member 3 further has a second engaging portion 33 that is detachably coupled to the first engaging portion 24. In this embodiment, the first engaging portion 24 is formed with a plurality of grooves 241 indented from the light incident face 21. The second engaging portion 33 is mounted on an end of the coupling sleeve 34 that faces the light conduction member 2, and includes a plurality of protruding blocks 331 for being respectively engaged with the grooves 241. Through the engagement design of the grooves 241 and the protruding blocks 331, the light conduction member 2 is detachably coupled to the light modulation member 3.

It is noted that the design of formation of the grooves 241 in the first engaging portion 24 and formation of the protruding blocks 331 in the second engaging portion 33 are interchangeable. In other words, either one of the first engaging portion 24 and the second engaging portion 33 may include the protruding blocks 331, while the other may include the grooves 241 for being engaged with the protruding blocks 331. Moreover, since the light modulation member 3 may be disposed proximate to the light exiting face 22 of the light conduction member 2, the first engaging portion 24 may be disposed proximate to the light exiting face 22 with the second engaging portion 33 being correspondingly disposed at a side of the light modulation member 3 that faces the light conduction member 2 for being engaged with the first engaging portion 24.

In this embodiment, since the light conduction member 2 and the light modulation member 3 are detachably coupled to each other, the combination of the light conduction member 2 and the light modulation member 3 is flexible and unrestrictive. For example, one light conduction member 2 is able to be coupled to various light modulation members 3, each of which has the second engaging portion 33 engageable with the first engaging portion 24 of the light conduction member 2, for achieving the intended light output power modulation.

It is noted that the light conduction member 2 may be configured as a combination of at least two components as shown in this embodiment, or may be alternatively configured as a one-piece component. In this embodiment, the light conduction member 2 includes a coupling body 25 that is formed with the first engaging portion 24 and a light guide body 26 that is securely assembled with the coupling body 25 and that extends away from the light modulation member 3. Similarly, the light modulation member 3 may be configured as a one-piece component or a combination of at least two components.

Referring to FIG. 8, the fourth embodiment of the present disclosure has a structure substantially the same as that of the third embodiment except that the first engaging portion 24 of the light conduction member 2 and the second engaging portion 33 of the light modulation member 3 are threadedly engaged in the fourth embodiment.

It is noted that the light modulation member 3 may be detachably connectable to the light exiting face 22 (see FIG. 1) of the light conduction member 2 with the first engaging portion 24 disposed on the light exiting face 22. Under this situation, the light emitted from the coherent light source 1 (see FIG. 2) passes through the light incident face 21 and the light exiting face 22 of the light conduction member 2 in sequence, and then passes through and is diffracted by the light modulation member 3 with the microstructure 30. Thereby, the light emitted from the optical needle assembly of such arrangement still has a modulated light output power distribution.

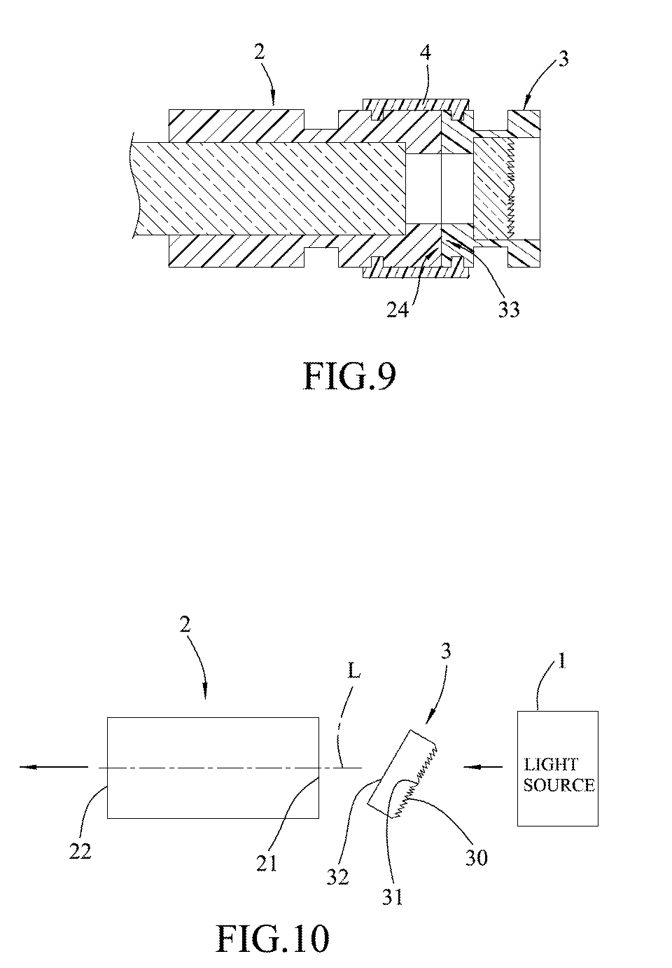

Referring to FIG. 9, the fifth embodiment of the present disclosure has a structure substantially the same as that of the third embodiment except that none of the grooves 241 and the protruding blocks 331 (see FIG. 6) are formed in either of the first and second engaging portions 24, 33 in the fifth embodiment. Instead, a peripheral sleeve 4 is further included to sleeve around peripheries of the first engaging portion 24 and the second engaging portion 33 so as to detachably couple the light conduction member 2 to the light modulation member 3.

Referring to FIG. 10, the sixth embodiment of the present disclosure has a structure substantially the same as that of the first embodiment except that the light modulation member 3 of the sixth embodiment includes a first face 31 and a second face 32 that are opposite to each other. The microstructure 30 is formed on at least one of the first and second faces 31, 32. In this embodiment, the first face 31 faces the coherent light source 1, the second face 32 faces the light incident face 21 of the light conduction member 2, and the microstructure 30 is disposed on the first face 31. It is noted that when the light modulation member 3 is disposed at the side of the light exiting face 22, the second face 32 faces the light exiting face 22 and the first face 31 is adapted to be formed with the microstructure 30. At least one of the first face 31 and the second face 32 of the light modulation member 3 is inclined relative to the central axis (L). The light emitted from the coherent light source 1 travels inside the light conduction member 2 in the direction of the central axis (L). This embodiment is likewise a transmission-type design. The inclination of the at least one of the first and second faces 31, 32 relative to the central axis (L) effectively reduces back reflection of the light upon the light incident face 21, the light exiting face 22, the first face 31 and the second face 32.

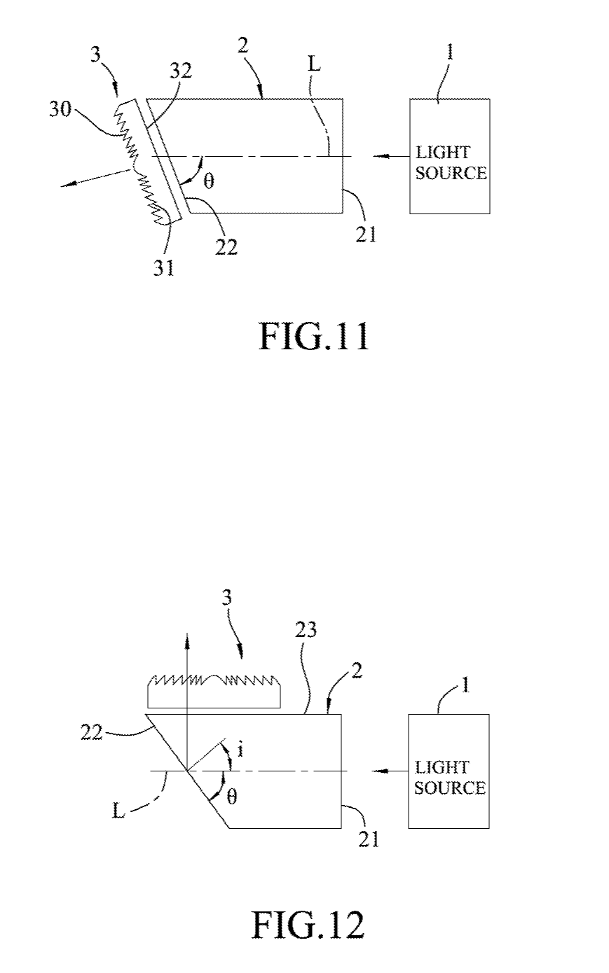

Referring to FIG. 11, the seventh embodiment of the present disclosure has a structure substantially the same as that of the first embodiment except that in the seventh embodiment, the light emitted from the coherent light source 1 travels inside the light conduction member 2 in the direction of the central axis (L) of the light conduction member 2, and that the light exiting face 22 of the light conduction member 2 is inclined relative to the central axis (L). An included angle (θ) formed between the light exiting face 22 and the central axis (L) is an acute angle larger than (90−θ_{c}) degrees and smaller than 90 degrees, where θ_{c} represents a critical angle for occurrence of total internal reflection of the light incident upon the light exiting face 22. The refraction index of the light conduction member 2 is larger than that of the medium outside the light conduction member 2. The light modulation member 3 is disposed proximate to the light exiting face 22 for the central axis (L) to extend therethrough. The light has a transmission direction inclined relative to the central axis (L) after being refracted from the light exiting face 22 and then passing through the light modulation member 3.

Therefore, in this embodiment, the light exiting the light exiting face 22 of the light conduction member 2 is refracted to be deviated from and inclined to the central axis (L). Then the inclined emission light is diffracted by the light modulation member 3 for conducting the light output power modulation. Hence, the transmission direction of the light emitted from the light field-modulable optical needle assembly of this disclosure is not limited to the linear direction as shown in the first embodiment, and may alternatively be guided to the inclined direction relative to the central axis (L). Thus, the emission directions of the light can be adjusted for rendering different appropriate emitting angles to be adapted for, for instance, treating different portions inside an oral cavity. Hence, the light field-modulable optical needle assembly is very convenient in use.

Referring to FIG. 12, the eighth embodiment of the present disclosure has a structure substantially the same as that of the seventh embodiment except that in the eighth embodiment, the included angle (θ) formed between the light exiting face 22 of the light conduction member 2 and the central axis (L) is larger than 0 degrees and smaller than (90−θ_{c}) degrees, and that the light modulation member 3 is disposed above the surrounding face 23, proximate to the light exiting face 22 and spaced apart from the central axis (L).

With respect to light emission, this embodiment belongs to a reflection-type design. The light emitted from the coherent light source 1 enters the light incident face 21 of the light conduction member 2 and propagates to the light exiting face 22. Due to the design of the included angle of (θ), the included angle (i) formed between the incident direction of the light upon the light exiting face 22 and the central axis (L) is larger than (θ_{c}) degrees and thereby a total internal reflection is resulted. In such a manner, the light is reflected by the light exiting face 22, passes through the surrounding face 23, and finally exits with a light output power distribution to be subsequently adjusted by the light modulation member 3. In this embodiment, the light is reflected by the light exiting face 22 of the light conduction member 2, and then, after passing the light modulation member 3, the transmission path of the light is inclined relative to the central axis (L).

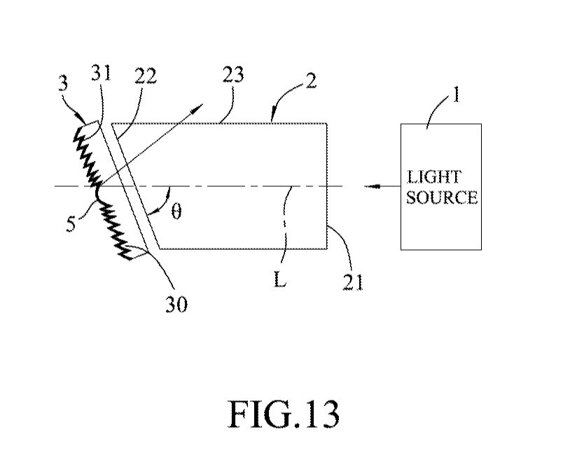

Referring to FIG. 13, the ninth embodiment of the present disclosure has a structure substantially the same as that of the seventh embodiment except that the light modulation member 3 further includes a reflective film 5. The reflective film 5 is disposed on a side of the light modulation member 3 distal from the light conduction member 2. In this embodiment, the reflective film 5 is disposed on the first face 31. After passing through the light exiting face 22 of the light conduction member 2 and the light modulation member 3, the light is reflected by the reflective film 5, and the reflected light will exit the surrounding face 23 of the light conduction member 2. In such a manner, after passing through the light exiting face 22 of the light conduction member 2 and the light modulation member 3 and being reflected by the reflective film 5, the transmission path of the light is inclined relative to the central axis (L).

For instance, the reflective film 5 may be coated on the light modulation member 3 by sputtering. The light reflectivity of the reflective film 5 ranges from 20% to 100%. The light not reflected by the reflective film 5 continuously propagates along the originally refracted light transmission path.

In view of the foregoing, the arrangement of the light modulation member 3 is flexible and may be disposed proximate to one of the light incident face 21, the light exiting face 22 and the surrounding face 23 of the light conduction member 2. The light emitted from the coherent light source 1 may pass through the light modulation member 3 before passing through the light conduction member 2, or alternatively, may pass through the light conduction member 2 before passing through the light modulation member 3. By disposing the light modulation member 3 proximate to the light incident face 21, the microstructure 30 is advantageously protected and not harmed during the surgery. By disposing the light modulation member 3 proximate to the light exiting face 22, the design of the light modulation member 3 is advantageously flexible since the light exiting side of the light modulation member 3 is free of spatial restriction. For example, the size of the light modulation member 3 is adjustable such that the first face 31 has a relatively large area for the microstructure 30, and the area of the microstructure 30 and the area of the first face 31 are made to be larger than that of the light incident face 21 or the light exiting face 22 of the light conduction member 2. The connection between the light conduction member 2 and the light modulation member 3 may be fixed or detachable. The light conduction member 2 and the light modulation member 3 may be integrally formed. The light may exiting the light field-modulable optical needle assembly in different directions or from different parts of the assembly through various arrangements of parts and use of varying optical path guiding means.

Moreover, through the corporation between the light conduction member 2 and the light modulation member 3, light spots of different shapes may be formed. For instance, when corporating with an appropriate light modulation member 3, a cylindrical light conduction member 2 may generate a linear or square light spot. In addition, for the conventional optical needle device, to acquire an inclined light emitting angle, the light conductive components have to be cut to form many special cutting angles. On the other hand, in this disclosure, intended refraction or reflection is achievable by employing the light modulation member 3. Therefore, the manufacturing procedures are significantly simplified. Compatibility between the light conduction member 2 and the light modulation member 3 of different specifications and shapes may provide users with more variations and flexibilities in applications, such as flexible arrangements of the size and geometric shape of light spots, the light output power distribution, the angles of light beams, etc. The light field-modulable optical needle assembly of this disclosure is able to meet every sort of characteristics of the light beams needed in various phototherapy involving laser or LED light, such as dental treatments directed to periodontal diseases, inflammation of dental peripheral explants, etc.

While the disclosure has been described in connection with what are considered the practical embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A light field-modulable optical needle assembly adapted for conducting a phototherapy, comprising
a coherent light source;
a light conduction member that has a light incident face which is proximate to said coherent light source, a light exiting face which is opposite to said light incident face and distal from said coherent light source, and a surrounding face which peripherally extends from said light incident face to said light exiting face to be connected therebetween; and
a light modulation member that is disposed proximate to one of said light incident face, said light exiting face and said surrounding face of said light conduction member, said light modulation member being formed with a microstructure,
wherein when light is emitted from said coherent light source, the emitted light enters and exits said light conduction member and said light modulation member so as to have a light output power distribution adjusted by said microstructure of said light modulation member.

2. The light field-modulable optical needle assembly as claimed in claim 1, wherein said light modulation member has a first face and a second face that are opposite to each other, said microstructure being disposed on at least one of said first and second faces, said light conduction member having a central axis, the light emitted from said coherent light source propagating inside said light conduction member in the direction of the central axis, at least one of said first and second faces being inclined relative to the central axis.

3. The light field-modulable optical needle assembly as claimed in claim 1, wherein said light modulation member is disposed on said light exiting face of said light conduction member, said light modulation member including a reflective film for reflecting the light passing through said light conduction member.

4. The light field-modulable optical needle assembly as claimed in claim 1, wherein said light conduction member having a central axis, the light emitted from said coherent light source propagates inside said light conduction member in the direction of the central axis, an included angle formed between said light exiting face of said light conduction member and the central axis being an acute angle larger than (90−θ_{c}) degrees and smaller than 90 degrees, where θ_{c} represents a critical angle for occurrence of total internal reflection of light incident upon said light exiting face, said light modulation member being disposed proximate to said light exiting face for the central axis to extend therethrough, the light that is refracted by said light exiting face and then passes through said light modulation member having a propagating direction inclined relative to said central axis.

5. The light field-modulable optical needle assembly as claimed in claim 1, wherein said light conduction member has a central axis, the light emitted from said coherent light source propagating inside said light conduction member in the direction of the central axis, an included angle formed between said light exiting face of said light conduction member and the central axis being an acute angle larger than (90−θ_{c}) degrees and smaller than 90 degrees, where θ_{c} represents a critical angle for occurrence of total internal reflection of light incident upon said light exiting face, said light modulation member being disposed above said surrounding face, proximate to said light exiting face and spaced apart from the central axis, the light that is refracted by said light exiting face and then passes through said light modulation member having a propagating direction inclined relative to said central axis.

6. The light field-modulable optical needle assembly as claimed in claim 1, wherein said light conduction member has a central axis, the light emitted from said coherent light source propagating inside said light conduction member in the direction of the central axis, an included angle formed between said light exiting face of said light conduction member and the central axis being an acute angle larger than (90−θ_{c}) degrees and smaller than 90 degrees, where θ_{c} represents a critical angle for occurrence of total internal reflection of light incident upon said light exiting face, said light modulation member being disposed proximate to said light exiting face for the central axis to extend therethrough, said light modulation member including a reflective film, the light that is refracted by said light exiting face, passes through said light modulation member and is reflected by said reflective film having a propagating direction inclined relative to said central axis.

7. The light field-modulable optical needle assembly as claimed in claim 1, wherein said coherent light source has a central wavelength, said microstructure including alternate crests and troughs and having structural pitches, each of which is defined by any two adjacent ones of said crests, said structural pitches ranging from 0.5 to 200 times of the central wavelength of said coherent light source.

8. The light field-modulable optical needle assembly as claimed in claim 1, wherein said coherent light source has a central wavelength, said light conduction member having a central axis, said microstructure including alternate crests and troughs and having structural heights, each of which is defined by a distance between one of said troughs to an adjacent one of said crests in the direction of the central axis, said structural heights ranging from 0.1 to 2000 times of the central wavelength of said coherent light source.

9. The light field-modulable optical needle assembly as claimed in claim 1, wherein a ratio of a projected area of said microstructure on a plane parallel to one of said light incident face and said light exiting face of said light conduction member relative to an area of said one of said light incident face and said light exiting face ranges from 10% to 200%.

10. The light field-modulable optical needle assembly as claimed in claim 1, wherein said light conduction member and said light modulation member are integrally formed, said microstructure of said light modulation member being disposed at one of said light incident face and said light exiting face of said light conduction member.

11. The light field-modulable optical needle assembly as claimed in claim 1, wherein said light conduction member includes a first engaging portion disposed proximate to one of said light incident face and said light exiting face, said light modulation member including a second engaging portion detachably coupled to said first engaging portion.

12. The light field-modulable optical needle assembly as claimed in claim 11, wherein one of said first engaging portion and said second engaging portion is formed with a protruding block, and the other formed with a groove for being engaged with said protruding block.

13. The light field-modulable optical needle assembly as claimed in claim 11, wherein said first engaging portion and said second engaging portion are threadedly engaged.

14. The light field-modulable optical needle assembly as claimed in claim 1, wherein said light conduction member includes a first engaging portion disposed proximate to one of said light incident face and said light exiting face, said light modulation member further including a second engaging portion proximate to said first engaging portion, said light field-modulable optical needle assembly further including a peripheral sleeve for detachably sleeving around said first engaging portion and said second engaging portion so as to detachably couple said light conduction member to said light modulation member.

15. The light field-modulable optical needle assembly as claimed in claim 1, wherein the light emitted from said coherent light source has a light output power distribution that conforms with the Gaussian distribution before passing through said light modulation member and that is modulated to conform with a flat-top distribution after passing through said light modulation member.

16. The light field-modulable optical needle assembly as claimed in claim 1, wherein said coherent light source has a central wavelength that ranges from 800 nm to 3000 nm.
